(19) United States
(12) Patent Application Publication
(10) Pub. No.: US 2004/0131500 A1
(43) Pub. Date: Jul. 8, 2004

(54) Device and method for evaluating platelets

(76) Inventor: Herbert S. Chow, San Diego, CA (US)

Correspondence Address: Ronald E Perez, Fulwider Patton Lee & Utecht, Tenth Floor, 6060 Center Drive, Los Angeles, CA, 90045, US

(21) Appl. No.: 10/466,657

(22) PCT Filed: Jan. 18, 2002
(86) PCT No.: PCT/US02/04545

(51) Int. Cl. G01N 33/00

(52) U.S. Cl. 422/72; 436/45

(57) ABSTRACT

A method and apparatus are disclosed for measuring platelet activation in a whole blood matrix under physiological conditions and independent of the activation of a coagulation cascade in the plasma. The method and system measure the propensity of cells in a biological fluid to form adherent cell aggregates and thrombin. The method accommodates both citrated and native venous blood. The system includes a simple, low-cost, single-use thrombogenic test cartridge for measuring the intensity of thrombus formation, and for providing quantitative measurement of platelet functions in a near-patient environment. Hemostatic activity of platelets in a biological fluid sample is assessed by monitoring the degree of adherent thrombus formation after cells are exposed to platelet agonists in a shear stress environment. The system may be configured to monitor the therapeutic effects of anti-platelet agents, to assess primary hemostatic response of platelets, and to assess thrombotic potential of platelets.

### CROSS-REFERENCE TO RELATED APPLICATION

This application is a national stage filing under 35 U.S.C. §371 and claims priority to PCT/US02/04545 entitled “Device and Method for Evaluating Platelets,” filed Jan. 18, 2002, which claims priority to U.S. Provisional Application No. 60/281,175 entitled “Cell Activation Device,” filed Apr. 2, 2001, and to U.S. Provisional Application No. 60/262,806 entitled “Thrombogenic Assay,” filed, Jan. 19, 2001, each of which are incorporated herein by reference in their entirety.

### BACKGROUND OF THE INVENTION

The present invention relates to analytical devices and methods for monitoring thrombus formation. These devices and methods provide reliable detection of platelet and leukocyte activation in biologic fluids including a biologic sample. More specifically, the present invention provides functional test cartridges and assays for detecting the potential of platelets and leukocytes to form thrombi in a whole blood matrix after exposing to shear stress. The present invention further relates to a device that activates cells in biological samples wherein a shear stress is applied to a portion of the sample. The invention also relates to an apparatus and method for determining the propensity for activated cells to adhere, aggregate, and form thrombi. The present invention further pertains to the diagnosis of disorders related to primary hemostasis, diseases related to congenital platelet disorders, acquired disorders such as ischemic stroke and myocardial infarction. Furthermore, the present invention is directed to providing a drug discovery platform for monitoring inhibitory effects of anti-platelet or anti-coagulation agents.

Compelling scientific and clinical data have clearly indicated that platelets and leukocytes play a central role in the development of Acute Myocardial Infarction (AMI) and ischemic stroke. Activation of platelets, leukocytes and the coagulation system is the direct result of endothelial cell injury due to elevated shear stress on the local diseased blood vessel. Shear stress increases the frequency of collision between platelets, and leukocytes and causes rupturing of atherosclerotic plaque and direct injury to the endothelial lining of the vessel wall. Shear forces may directly activate platelets and leukocytes even when their exposure to such forces is brief. It has been shown that shear stress alone without the addition of an exogenous agonist can induce platelet and leukocyte aggregation in a manner very similar to that observed on an injured arterial wall. Shear stress of blood may exceed seventy dyne/cm² in partially occluded arteries by atherosclerosis or vascular spasm. In pathologic blood vessels, wall shear rates can well exceed one hundred dyne/cm². High shear stress not only causes platelet activation, accumulation, aggregation and adhesion, but also causes leukocytes to adhere to activated platelets through p-selectin and CD11b/CD18 receptors. These conditions create an environment conducive to local thrombus formation in the diseased arteries. The propensity to form adherent thrombi is directly proportional to the thrombotic risks.

### Platelet Function Tests

Platelet aggregation studies based on turbidimetric measurement have been used at least since the mid-1970's to estimate blood platelet functions in isolated platelets or whole blood matrix. A wide variety of other laboratory devices have been used to monitor the functional properties of platelets. A number of devices, discussed herein, have been disclosed for analyzing biological fluid samples under shear conditions.

More recently, a method was disclosed that improved over the conventional “Cone & Plate Viscometer.” That method provides a continuous measurement of isolated platelets in a suspension to which a shear force has been applied. The shear-induced platelet adhesions occur on the surface of the test device, and are measured by a transmitted ray detector unit. Such test methods and the conventional turbidimetric aggregometry require the use of isolated platelets, and conduct platelet evaluation under non-physiological conditions. Also known within the art of biological assays is a shear method that measures the adhesion of platelets to immobilized agonists in anti-coagulated blood. The rate of platelet response is measured by optical means. Since the effect of anti-coagulation is not neutralized, the test is not carried out under physiological conditions.

One known method that incorporates the induction of shear stress includes utilizing devices that measure the clot elasticity modulus resulting from the actions of platelets during clot formation and dissolution. In addition, methods have been described that measure the contribution of platelets on activated clot time. Such clotting tests are based on viscosity or fluidity changes when a fluid sample changes from a liquid to a gel form. It is believed that such a change is due to the conversion of soluble fibrinogen to insoluble fibrin by the action of the enzyme thrombin. Those tests detect changes in the viscosity or fluidity of the blood sample and present the results in the form of a clotting time. One drawback of such a method is that the activated clot time is not a platelet-specific event, since it represents the composite activity of platelet activation and the activation of the intrinsic or extrinsic coagulation pathways.

Also known in the art is a shear method that is capable of determining cell aggregation in anti-coagulated whole blood in response to agonists. The method uses an external pump that mixes the sample and controls its fluid velocity. The formation of platelet aggregates is detected by light scattering using a coherent light source. After analysis, the used vessel and the blood sample sealed within are discarded without the risk of making contact with the operator. The single-use disposable allows the method to be used in routine clinical settings. Two drawbacks of the method are that it is not conducted under physiological conditions and that the method requires complex instrumentation similar to many of the previous methods described heretofore.

A simple disposable aspiration device has been disclosed that directs blood flow into a reservoir through a capillary tube. Thrombus formation occurs in the capillary tube solely by the flow-generated shear forces. The volume of blood collected in the reservoir within some predetermined time period correlates with the formation of thrombi in the capillary and is indicative of platelet activity in the blood sample. The method can be carried out on samples of native venous blood. The drawback of such a method is that it provides only a semi-quantitative estimation of the thrombotic risks of platelets. The result is not specific to platelet activation, since it represents the composite activity of platelet and the activation of the intrinsic or extrinsic coagulation pathways.

### Shear Stress Generation

Atherosclerosis is a geometrically focal disease, preferentially affecting the outer edge of vessel bifurcations, especially in areas where blood shear forces are weak and non-laminar (i.e., turbulent) flow dynamic occurs. As the buildup of complex plaque material progresses, the irregular geometry of the vessel wall and the narrowing of the vessel lumen induce changes in the shear rate and fluid dynamics of flowing blood. The severity and quantity of thrombus formation increases as shear force and turbulence flow increases. The transition from a high-shear, accelerating flow at the point of stenosis to a downstream region where blood components are thereby exposed to decelerating forces and the formation of larger static pools of re-circulation regions is a unique fluid characteristic of the diseased artery. Activation of platelets and leukocytes may occur at the accelerating stream, but thrombus formation tends to proceed more rapidly in low shear stress areas, such as vessel bifurcation points.

Shear devices have been disclosed that deliver a laminar shear force on the fluid sample, for example, a motor may be used to drive a rotational unit. Alternatively, a magnetic element is embedded in the rotational unit, and its motion is controlled by means of magnetic coupling to an external magnetic driver. There are a few drawbacks with such shear devices. Those devices are not designed to be disposable, and the biological sample in the device may come into contact with the operator. The design of a circular rotor rotating inside a uniformly cylindrical housing generates laminar flow, and does not reflect the fluid dynamic of a blocked artery.

### Transparent Test Device for Biologic Fluid

A variety of optical illumination methods are available to quantitatively or qualitatively monitor events and analytes or measure analyte concentration in biological fluid contained inside a transparent test device. In a transmissive optical method, one or more illuminators are typically located on one of the external surfaces of a transparent or translucent test device containing a biological fluid. Additionally, one or more detectors are placed adjacent to the opposite external surface of the device. In a transmissive optical method, a ray of light is passed through the biologic fluid. As the ray passes through the fluid, it decreases in intensity, indicating events or concentrations of analytes in the fluid. The illuminator(s) may utilize ray spectra ranging from infrared to ultraviolet, including the visible light spectrum.

The prior art also contains various designs of transparent analytical devices that contain a multitude of capillary passageways for moving biological fluid by means of capillary forces. A reagent contained within the passageway interacts with blood and causes changes in fluidity as clots form when blood transforms from a solid to a gel state. There are drawbacks to such designs and methods of use. During blood coagulation, other erythrocyte aggregates passively trapped in the clot cannot be distinguished from platelet aggregates or platelet-leukocyte hetero-aggregates. Furthermore, adherent aggregates cannot be distinguished from non-adherent clusters formed between erythrocytes, platelets and/or leukocytes.

In view of the forgoing, it would be an advance in the art to provide a method and apparatus for measuring platelet activation in a whole blood matrix under physiological conditions. There is a need to provide a method that can accommodate both citrated and native venous blood. It would also be an advance in the art to provide an improved method of assessing platelet activation independent of the activation of a coagulation cascade in the plasma. Such a test should capture the properties of activated platelets to aggregate, adhere, and form thrombi under the influence of shear stress. In light of the physiology of the stenotic vessel, it would be an advance in the art to design a shear device that generates a non-laminar fluid flow pattern and incorporates the acceleration and deceleration flow patterns that are characteristic of the stenotic artery. Existing systems for assessing platelet functions require complex mechanical design, repeated cleaning, and lack portability. It would be an advance in the art to provide a simple, low-cost, disposable device that would allow quantitative measurement of platelet functions in a near-patient environment. The system would be appropriate for detecting hyperactive platelets in patients with increased thrombotic risks, and for detecting platelet dysfunction in patients with primary hemostasis disorder. The present invention satisfies these and other needs.

### SUMMARY OF THE INVENTION

Briefly, and in general terms, the present invention is directed to the design and configuration of a single-use method and system that measures the propensity of cells in a biological fluid to form adherent cell aggregates and thrombin. The system consists of a thrombogenic test cartridge and an instrument that measures the intensity of thrombus formation. Hemostatic activity of platelets in a biological fluid sample can be assessed by monitoring the degree of thrombus formation after cells are exposed to known platelet agonists under low shear stress environment. Thrombotic potentials of platelets can be assessed after cells are exposed to moderate shear stress and thrombogenic substrates that are commonly encountered in athero-sclerotic plaque. Cells with an inherently higher baseline thrombus activity can be evaluated in the thrombogenic test device without first being activated by the shear device. Apparatuses for performing the methods of the present invention are disclosed herein. The system can also be used to monitor therapeutic effects of anti-platelet agents, to assess primary hemostasis response of platelets, and to assess thrombotic potential of platelets.

Objectives of the Standalone Turbulent-Shear Device (TSD)

(a) Provide a device that generates variable shear forces.

(b) Provide a bearing-less and seal-less shear device that eliminates heat generation from bearings, leakage of seals, dead volume zones and low efficiency.

(c) Provide a shear device that mimics the unique characteristics of blood flow in a stenotic artery.

(d) Provide a single-use shear device with improved safety of use while simplifying its construction and reducing cost.

Objectives of the Standalone Thrombogenic Test Device (TTD)

(a) Provide a transparent laminated test device for evaluating cell activity in a biological fluid sample to form adherent aggregates and micro-thrombi in response to various platelet agonists under low shear stress and low flow environment.

(b) Provide dry reagents that can be used to assess platelet response in primary hemostasis.

(c) Provide dry reagents that mimic the activity of thrombogenic substances in atherosclerotic plaque to assess thrombotic potential of platelets.

Objectives of the Thrombogenic Test Device (TTD) When Integrated with the Turbulent-Shear Device (TSD)

(a) Provide a thrombogenic test cartridge with integrated shear device that measures the ability of biological fluid to form adherent aggregates and micro-thrombi in moderate to high shear stress and flow environment.

(b) Provide a method to fabricate the thrombogenic test cartridge.

Objectives of the Thrombogenic Test System

(a) Provide a method to separate adherent aggregates and thrombi from non-aggregated particles prior to the full activation of clot formation in plasma.

(b) Provide a system that minimizes contact between the operator and the biological sample and allows disposal of the self-contained test sample after a single use in laboratory or point-of-care settings.

(c) Provide a method to measure the light absorption of adherent thrombi in the light path over discrete time intervals. The absorption numbers provide the user with an indication of the intensity and size of the adherent thrombi.

(d) Provide a method to monitor inhibitory effects of pharmacological agents on platelet activation.

(e) Provide a method to measure platelet activity in primary hemostasis.

(f) Provide a method to measure thrombotic potential of platelets.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a side cross-sectional view of an assembled turbulent-shear device of the present invention, showing the components of FIGS. 2B, 3 and 4B.

FIG. 2A is a top plan view of one embodiment of the rotor of the turbulent-shear device of the present invention.

FIG. 2B is a side plan view of the rotor of the turbulent-shear device shown in FIG. 2A.

FIG. 3 is a side cross-sectional view of one embodiment of the sample-containing housing of the turbulent-shear device of the present invention.

FIG. 4A is a top plan view in partial cross-section of one embodiment of the top cover of the turbulent-shear device of the present invention.

FIG. 4B is a side plan view of the top cover of the turbulent-shear device shown in FIG. 4A.

FIG. 5 is a top plan view in partial cross-section of an assembled turbulent-shear device of the present invention, showing the components of FIGS. 2A, 3 and 4A.

FIGS. 6A, 6B and 6C are top plan views of alternate embodiments of the turbulent-shear device of the present invention.

FIG. 7 is a side plan view in partial cross-section of an embodiment of the turbulent-shear device of the present invention showing a transfer apparatus.

FIG. 8 is a top plan view of one embodiment of a thrombogenic test cartridge of the present invention.

FIG. 9 is a top plan view of one embodiment of a thrombogenic test unit of the present invention.

FIGS. 10A, 10B, 10C and 10D illustrate an alternate embodiment of the thrombogenic test cartridge of the present invention having integrated test units and turbulent shear devices with built-in flow control mechanisms.

FIG. 11 illustrates a manufacturing method of one embodiment of the test cartridge of the present invention integrated with multiple test units and shear devices.

FIG. 12 is an exemplary graph showing a typical light scattering result of objects in the view area of the test cartridge of the present invention.

FIG. 13 is a simplified block diagram illustrating a signal detection and processing method of the present invention.

FIG. 14 is a schematic representation of a simplified system arrangement illustrating an embodiment of the turbulent-shear device and the thrombogenic test cartridge of the present invention.

FIG. 15 is a schematic representation of a flow control mechanism of one embodiment of the test cartridge of the present invention.

FIG. 16A is a top plan view of another embodiment of the thrombogenic test cartridge of the present invention, wherein a flow control mechanism is integrated with the test unit.

FIG. 16B is a top plan view of the test unit and flow control mechanism shown in FIG. 16A.

FIG. 16C is a cross-sectional view of the test unit and flow control mechanism shown in FIG. 16B.

FIG. 16D is a side plan view of the deformable membrane of FIG. 16B in a deformed position.

FIG. 16E is a side plan view of the deformable membrane of FIG. 16B in a rest position.

FIG. 17 is a graph illustrating the intensity of thrombus formation in blood exposed to varying durations of a shear stress.

FIG. 18 is a graph illustrating the intensity of thrombus formation in blood exposed to varying shear rates.

FIG. 19 is a graph illustrating the system ability to detect platelet dysfunction in samples from patients with von-Willebrand Disease or Glanzmann Thrombasthenia.

FIG. 20 is a graph illustrating the inhibition of thrombus formation in the presence of antibodies to various ligands or coagulation factors (IgG—immunoglobulin; >vWF—antibody to von-Willebrand Factor, >FBG—antibody to fibrinogen; >CD41a—antibody to GPII receptor; >P-Sel—antibody to P-Selectin; >FVII—antibody to Factor VII; >TF—antibody to Tissue Factor; >FX—antibody to Factor X).

FIG. 21 is a graph illustrating the inhibition of thrombus formation in the presence of various anti-platelet or anti-thrombin agents.

### DETAILED DESCRIPTION OF THE PREFERRED EMBODIMENTS

As shown in the drawings for purposes of illustration, the present invention is directed to the design and configuration of a thrombogenic test system configured to provide a set of apparatus and several methods with which to measure the propensity of an activated blood sample to form adherent cell aggregates and thrombi. The apparatus includes two devices. The first device, the turbulent-shear device, is a single-use apparatus for generating non-laminar shear stress on biologic samples including whole blood. Platelet and leukocyte activation occurs as a result of exposure to shear stress. The second device, the thrombogenic test cartridge, is a single-use transparent fluid diagnostic device for measuring the properties of cells in a biological sample. The invention can be used to determine the thrombotic potential of cells in a biologic sample. The invention can be further used to study platelet dysfunction in primary hemostatic disorder. In a separate embodiment, the invention can also be used to study agents that can negatively or positively modulate the formation of adherent cell aggregates and thrombi in a blood sample.

Referring now to the figures, by way of example, the first embodiment of the thrombogenic test system, as shown in FIGS. 1-5, which comprises a turbulent-shear device 30 in a stand-alone fashion that generates non-laminar turbulent shear forces on a biological sample. The turbulent flow mimics the flow characteristic in a stenotic artery. Conversely, in a conventional cone and plate shear device, such as that known to one of ordinary skill in the art, a circular rotor rotates inside a cylindrical chamber containing a fluid sample. The side surface of such prior art rotors has a uniform curvature and the face opposite to the inner bottom of the rotor has a conical surface at an angle of three degrees or less. Such devices generate shear stress that is essentially laminar with a constant stress throughout the entire surface of the rotor.

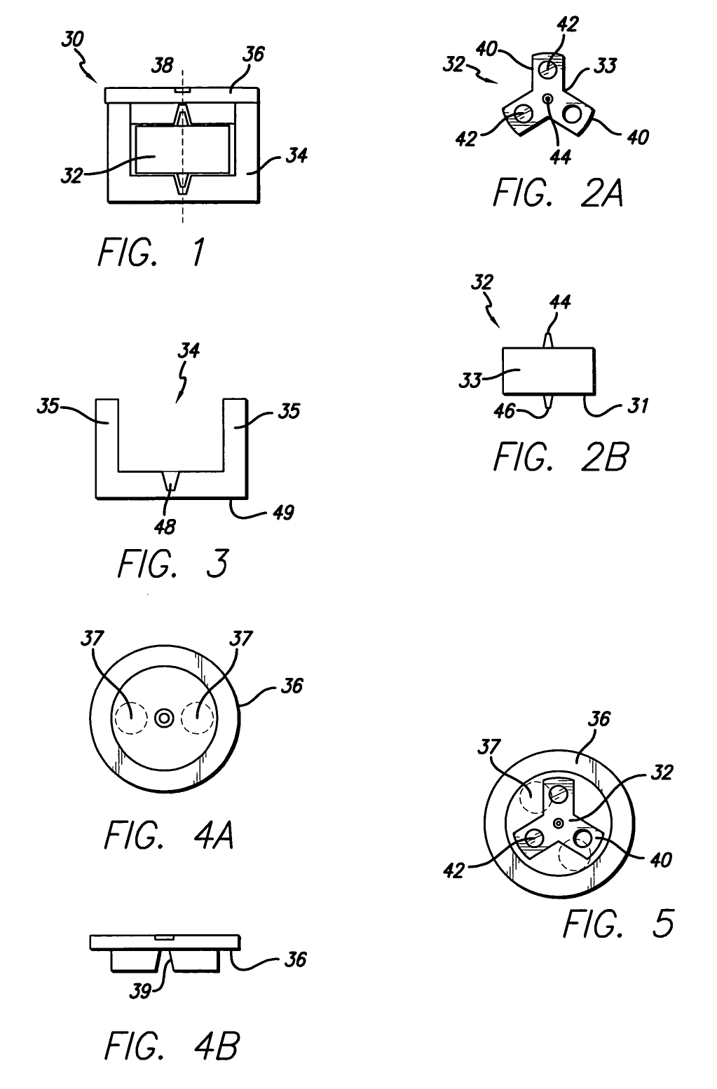

Referring to FIG. 1, one embodiment of the turbulent-shear device 30 of the present invention includes a cylindrical rotor 32 retained within a housing forming a sample chamber 34 comprising the lower portion of the turbulent-shear device. A top plate 36 comprises the upper portion of the housing, fitting within a wall 35 of the sample chamber (FIG. 3) and covering the rotor. The cylindrical rotor is configured with a non-uniform curvature on each side 33 (FIG. 2A), and with an inner bottom face 31 having a flat surface (FIG. 2B). The non-uniform curvature of the sides of the rotor provides turbulent flow with multiple acceleration and deceleration zones inside the sample chamber.

The cylindrical rotor 32 may be suspended between the top plate 36 and the wall 35 of the sample chamber 34 such that the rotor rotates freely along the center axis 38 of the turbulent-shear device 30. The rotor may be configured with an upper flange 44 that rotatably fits with a fitting 39 in the top plate. Similarly, the rotor may be configured with a lower flange 46 that rotatably fits within a fitting 48 in the bottom of the sample chamber. The rotor, the sample-containing chamber (housing) and the top plate may be constructed from transparent and inexpensive resin for easy direct viewing. Both the cylindrical rotor and the sample container are preferably formed from non-magnetized materials.

As shown in FIG. 2A, the rotor 32 may be configured with one or more arms 40 (for example, three arms), such that one or more of the rotor's arms (for example, two arms) are metalized (contains magnetizable material) or embedded with small magnetizable metal pieces 42. As a result, the inner surface 31 of the cylindrical rotor facing the bottom of the sample housing 34 is asymmetrically metalized, such that the rotor can be driven by a radial magnetic field. Sources of a magnetic field may include a permanent magnet or electromagnet mounted on a rotating device in close proximity to the external bottom surface 49 of the sample container 34. The coupling between the asymmetrically metalized rotor and the radial magnetic field allows the construction of a bearing-less and seal-less shear device that eliminates heat generation from bearing movement and seal leakage. Other mechanical means, such as direct drive using a motor or pneumatic device (not shown), can also be used to couple the rotor and the external driver.

The turbulent-shear device 30 can be molded to handle small volumes of biological fluid. When required, the temperature of the biological sample can be maintained through an external heating element (not shown). This example of a standalone shear device preferably has at least one opening 37 in the top plate 36 that allows the transfer of fluid into and out of the device. The sheared sample can be manually retrieved from the device and analyzed in the thrombogenic test device or in other conventional analytical devices.

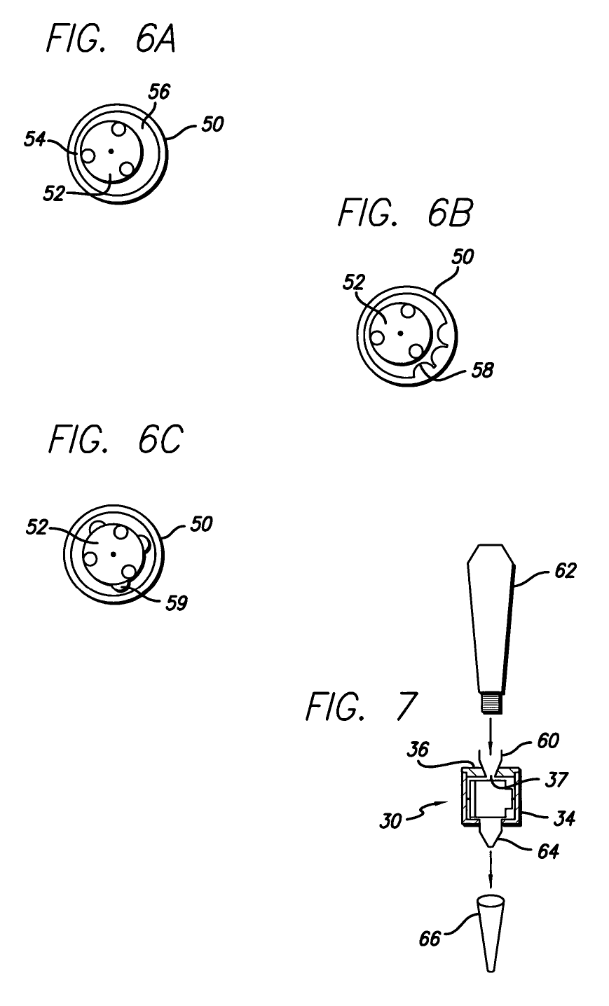

In alternate configurations of the turbulent-shear device, as shown in FIGS. 6A, 6B and 6C, the rotor 52 may be configured with an axis of rotation that is shifted off-center, such that a constant gap is no longer maintained between the inner wall of the sample container 50 and the side-wall of the rotor. As the fluid sample streams around the spinning rotor, flow accelerates as fluid enters the area with the narrowest gap 54 and decelerates as it leaves to the side with a wider gap 56 (FIG. 6A). Such alternate configurations may include a circular rotor and a circular sample chamber housing the rotor. Alternative configurations may include rotors and/or sample chambers with non-circular shapes. These configurations also generate non-laminar flow with single or multiple acceleration and deceleration zones. Multiple acceleration and deceleration zones can also be achieved by placing obstructions 58 in the flow path of the rotor (FIG. 6B). Flow acceleration and deceleration occur as fluid flows over these obstructions. In another configuration, obstructive objects 59 are placed on the surface of the rotor instead of on the surface of the housing (FIG. 6C). These objects create varying degrees of obstruction along the flow path. The construction of multiple acceleration and deceleration areas mimics the non-laminar blood flow characteristic of a stenotic vessel.

The current invention of the turbulent shear device as a single-use apparatus provides improved safety of use with simplified construction and reduced cost. The design allows the rotor to be driven by a rotating magnetic field in close proximity to the shear drive. Advantages resulting from this arrangement lie essentially in the fact that the dynamic balance of the rotator is greatly improved in comparison with prior art devices. This invention decreases the cost, size and sample volume of the shear device to a considerable extent and makes it practical as a disposable device.

Yet another alternative use of the turbulent shear device 30 is one that provides connectivity to external devices that can assist in fluid transfer. In the example in shown in FIGS. 1-5, the top plate 36 has at least one opening 37 to allow the transfer of fluid into and out of the device. In an alternate configuration as shown in FIG. 7, the top plate or sample housing 34 of the shear device is configured with a coupling feature (fitting) 60 that connects to a fluid transfer apparatus, such as a squeezable bulb 62. Another coupling feature (fitting) 64 may be formed in the housing (e.g., bottom side) to connect to a pipette tip or a blind cap 66. The fluid transfer apparatus provides the positive or negative pressure necessary to propel and aspirate the fluid, while the pipette tip provides the passage for fluid transfer into and out of the shear device.

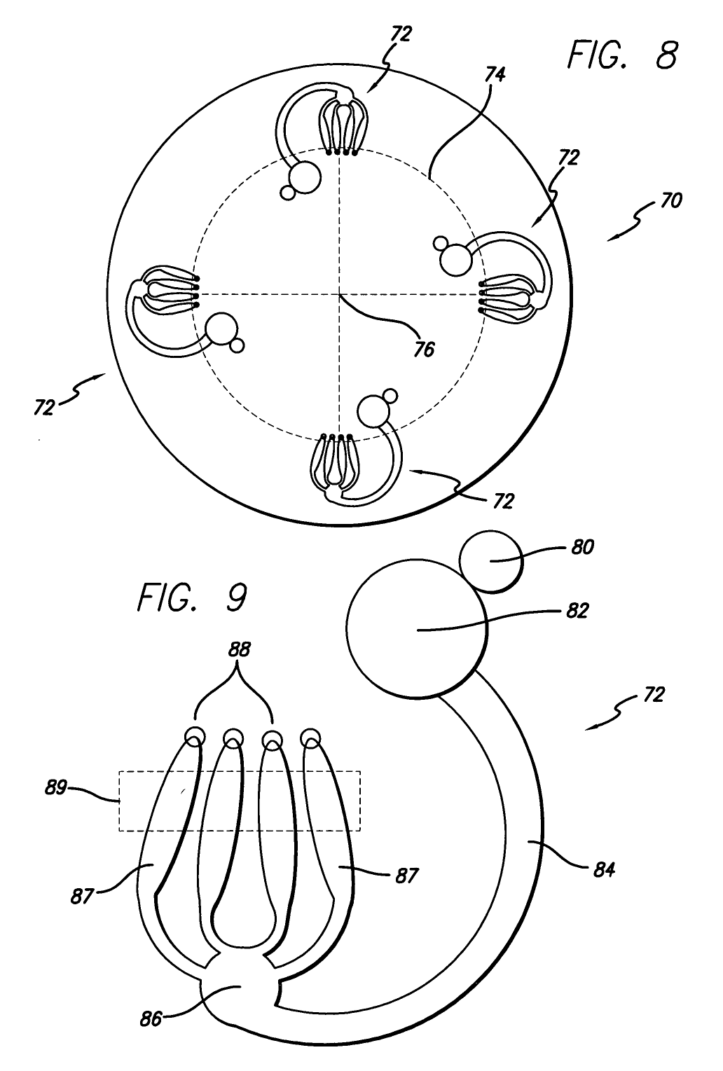

Referring now to FIGS. 8 and 9, the second embodiment of the Thrombogenic Test System comprises a thrombogenic test cartridge 70 that measures the propensity of activated cells in a whole blood sample to form adherent thrombi and/or cell aggregates. The test is performed in a single (not shown) or multi-test unit 72 transparent cartridge with the results viewed with a transmissive or reflective optical system with spectra in the visible, infrared or ultraviolet wavelengths. Each test unit may comprise of single (not shown) or multi-channel 87. In this embodiment of the present invention, channels are formed between two transparent plastic sheets held together by a third plastic sheet with double-sided adhesive. These plastic sheets have cutouts in various locations to form multiple transparent capillary channels. A reagent is pre-dried on the inner surface of the capillary channel and, upon contact with the blood sample, can cause blood cells to aggregate. The adherent property of cell aggregates causes them to bond with the inner surface of the capillary channel.

The thrombogenic test cartridge provides an in vitro diagnostic device for monitoring the properties of a biological fluid. The device comprises a first and second layer separated by an intermediate layer in which cutouts are formed and which is in contact with the first and second layers. With reference to FIG. 8, a test cartridge incorporating the present invention may consist of single or multiple test units arranged in a circular pattern 74 radiating from the center of rotation 76.

With reference to FIG. 9, each test unit 72 includes:

(a) An opening configured as an application site 80 for introducing a biological sample into the device;

(b) A reservoir 82 with a larger volume than the application area 80 for holding a blood sample;

(c) A first (transfer) channel 84 having a first and a second end, which provides a fluidic path from the reservoir 82 to a common area 86;

(d) Multiple reaction channels 87 that branch out from the common area 86 with each reaction channel terminating at a vent to the outside 88; and

(e) A viewing area 89 configured for providing analytical (optical reader) access to a portion of the reactions channels.

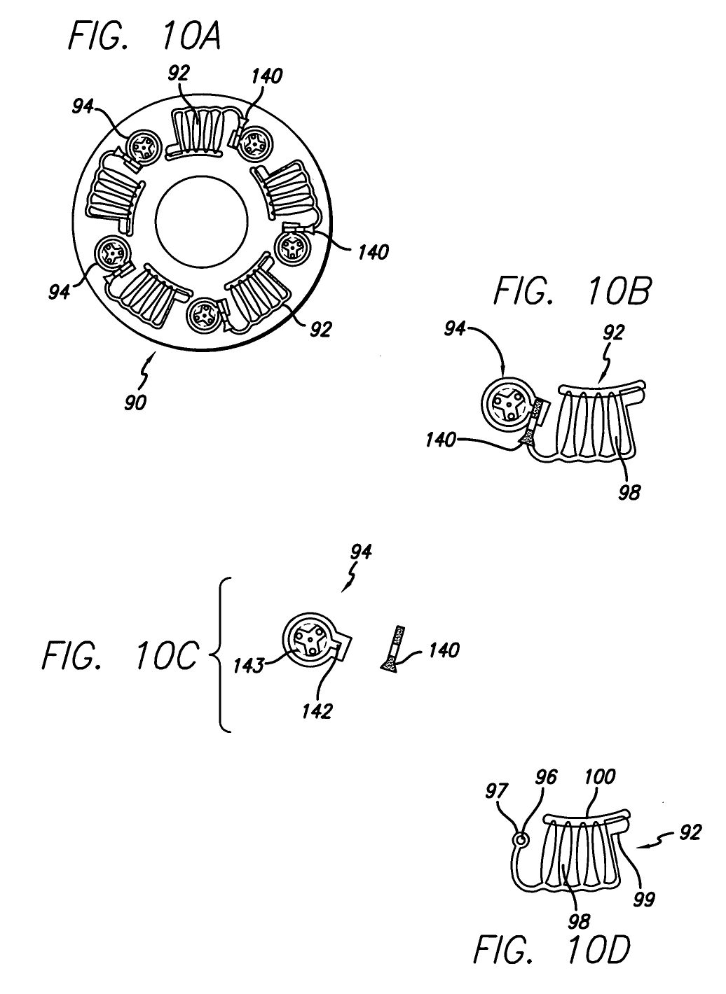

An alternate design for the thrombogenic test cartridge 90 is shown in FIGS. 10A-10D. In this embodiment, each test unit 92 includes an application site 96 in fluid communication with a plurality of reaction channels 98 (e.g., four reaction channels) and at least one by-pass channel 99 for flow control. The reaction channels 98 are arranged in a circular pattern radiating from the center of rotation. Both reaction channels and the by-pass channel terminate at a common vent 100 to the outside. Preferably, the application site 96 is positioned at the level of the vent 100.

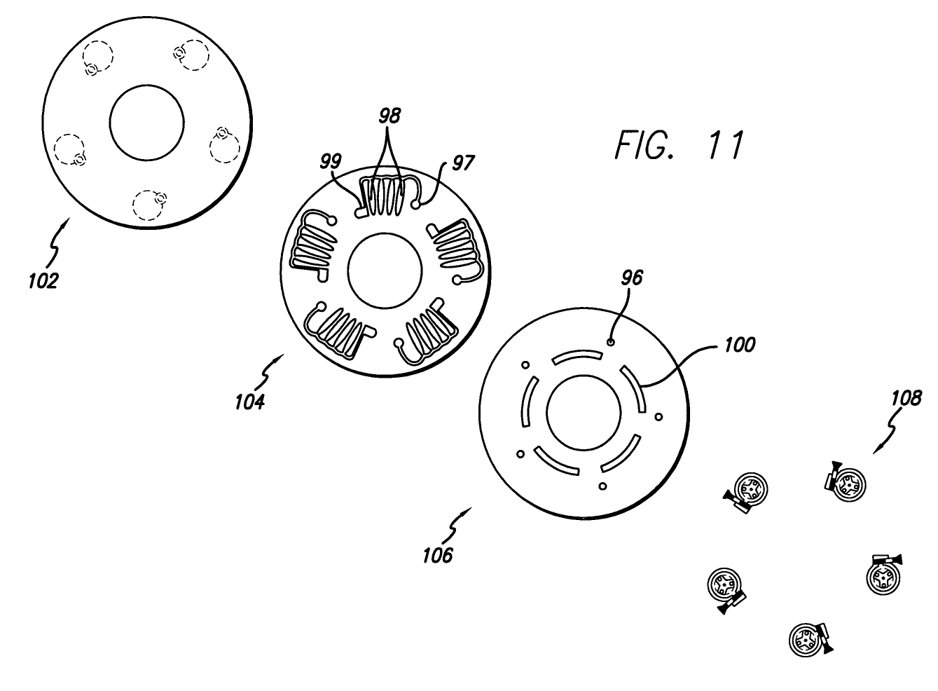

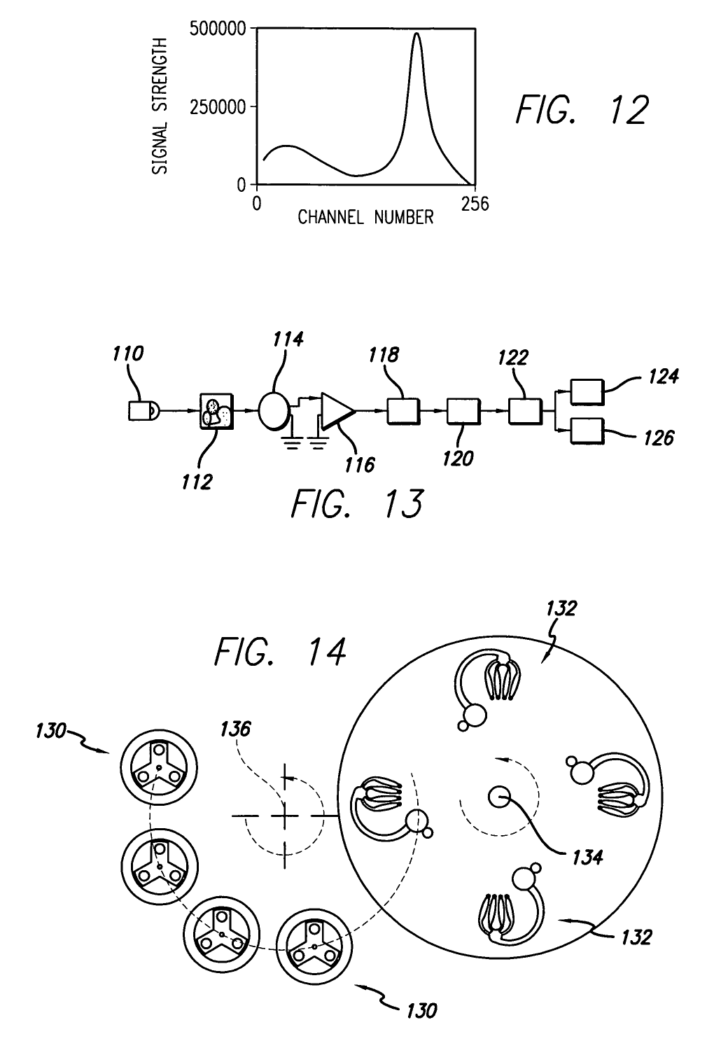

As shown in FIG. 11, all the elements described above are formed by cutouts in the middle layer 104 and the top layer 106 of a laminated test cartridge. Preferably, the middle layer 104 is formed from double-side adhesive tape; however, other materials may be used, as is well known to those of ordinary skill in the art. Layer 102 and 106 can be made out of hydrophobic and/or hydrophilic materials. The common vent 100 may be formed by a cutout in the top layer 106 and aligned with top end of the reaction channels 98 and the by-pass channel 99. The upper portion of the sample application site 96 may also be formed by a cutout in the top layer 106 and aligned with the remaining part of the sample application site 97 formed from a cutout in the middle layer. The size of the opening at the upper portion of the sample application site 96 may be smaller than the opening at the middle layer of the sample application site 97 to create a compartment for holding fluid. A plurality of shear devices 108 may be laminated on the surface of the top layer 106, using double-side adhesive tape or other suitable materials. The outflow from the shear device is aligned with the upper opening 96 of the sample application site to facilitate fluid transfer from the shear device to the test unit. Alternatively, the top layer 106 and its cutouts are provided in the form of a plastic housing. In such a configuration, the shear devices are designed as integral parts of the plastic housing, which provides a more rigid support and more secure mean to align the shear devices with the application sites on the test cartridge 90.

The third embodiment of the thrombogenic test cartridge provides means to contain biologic fluid inside the test system during operation. As a stand-alone device, such as the configuration shown in FIGS. 8 and 9, the test unit has a large reservoir 82 that serves to drain excess fluid sample away from the application site. An enclosed reservoir 82 is connected immediately down-stream from the sample application site 80. The reservoir has a larger fluid-holding volume than the sample application and allows the drainage of blood away from the application site, leaving the site with a minimum amount of fluid remaining after the filling process is complete. The containment of biological fluid inside an enclosed reservoir, along with leaving the opened sample application site void of biological fluid, serves to minimize the chances of the operator coming in contact with biological fluid. Furthermore, draining excess fluid from the application site may also minimize potential contamination of the instrument as well as the environment during the centrifugation step.

In a conventional capillary device, air bubbles trapped in a fluid sample could potentially migrate into the capillary channel during sample filling. These bubbles may distort the image in the viewing area, impede fluid flow, or compromise signal quality.

The fourth embodiment of the thrombogenic test cartridge provides a U-shape capillary channel 84 so that bubbles, if present, are always trapped or stay in close proximity to the application site 80 and the reservoir 82 and the descending portion of the channel 84. These measures prevent bubbles from entering the viewing area 87.

The fifth embodiment of the present invention combines the use of the thrombogenic test cartridge with an apparatus capable of generating centrifugal force. The present invention provides a method to detect the formation of adherent thrombi and cell aggregates in a biologic sample. While traditional coagulation assays can often be conducted in a cell-free environment, the formation of adherent thrombi in the present invention is dependent on the presence of platelets, leukocytes, and erythrocytes. After mixing blood with the platelet agonists or thrombogenic substances in the thrombogenic test cartridge and subsequent to a short incubation, the test cartridge is centrifuged at a speed sufficient to separate plasma from non-adherent cellular contents. In a positive reaction, adherent cell aggregates and thrombi can be readily detected in the plasma phase. Since the blood sample must maintain its fluidity and viscosity to allow separation of cells from plasma during the centrifugation step, events related to platelet-induced thrombi as described in the present invention are distinctly different from the clot formation due to activation of intrinsic or extrinsic coagulation pathways. Furthermore, in the present invention, platelet-induced adherent thrombi are induced prior to the critical change in blood viscosity that corresponds to the enzymatic degradation of fibrinogen and polymerization of fibrin in the plasma.

In the sixth embodiment, adherent cells are detected using a reflective or trans-missive light path through the view area on the thrombogenic test cartridge. FIG. 12 shows an exemplary graph of light scattering results of adherent cell aggregates through the view area. FIG. 13 shows a simplified system block diagram, illustrating the interpretation of the light scattering signal. The light source 110 transmits light through the view areas 112 on the test cartridge. Light scatter from the sample is measured by the photomultiplier tube (not shown) or photodetector 114, enhanced by the amplifier 116, and digitized by the analog to digital (A to D) converter 118. Histogram or peak/slope detection is conducted in block 120. The digitized information is stored in memory and processed in the microprocessor CPU 122. The results are either sent to a display device 124 or sent to other I/O devices 126.

In an alternate embodiment, contrasting materials such as dyes, fluorescent or luminescent materials or immuno-chemicals can be used to highlight cells or cell clusters in the whole blood.

Aside from using automated optical systems to visualize cell aggregates, other means for scoring thrombus formation may include direct microscopic observation with or without the aid of other analog or digital devices.

When shear device and thrombogenic test cartridge are both used as stand-alone apparatuses, they can be arranged to handle multiple tests of a sample or to simultaneously test multiple samples. In the seventh embodiment as shown in FIG. 14, a plurality of shear-generating devices 130 can be arranged in a circular path. After processing of the sample in the shear device, fluid samples are transferred from the shear unit to the sample application site of a pre-assigned test unit 132, also arranged in a circular path with a pre-configured first center of rotation 134. Fluid transfer is accomplished through a fluid-handling device (not shown) pivoting at a pre-configured second center of rotation 136. The timing, scheduling and transfer of fluid may be controlled by an on-board computer.

In the eighth embodiment as illustrated in FIGS. 10A-10D, the preferred embodiment of the test cartridge 90 is configured such that multiple test units 92 and a plurality of shear devices 94 are integrated on the test cartridge (FIG. 10A). A flow control mechanism (valve) 140 may be configured as part of either the shear devices or the test units (FIG. 10B). An opening 142 is formed in each shear device (exploded view FIG. 10C) to establish fluid communication (outlet or vent) between the inner chamber 143 of the shear device and the bottom layer 102 of the test cartridge (FIG. 11).

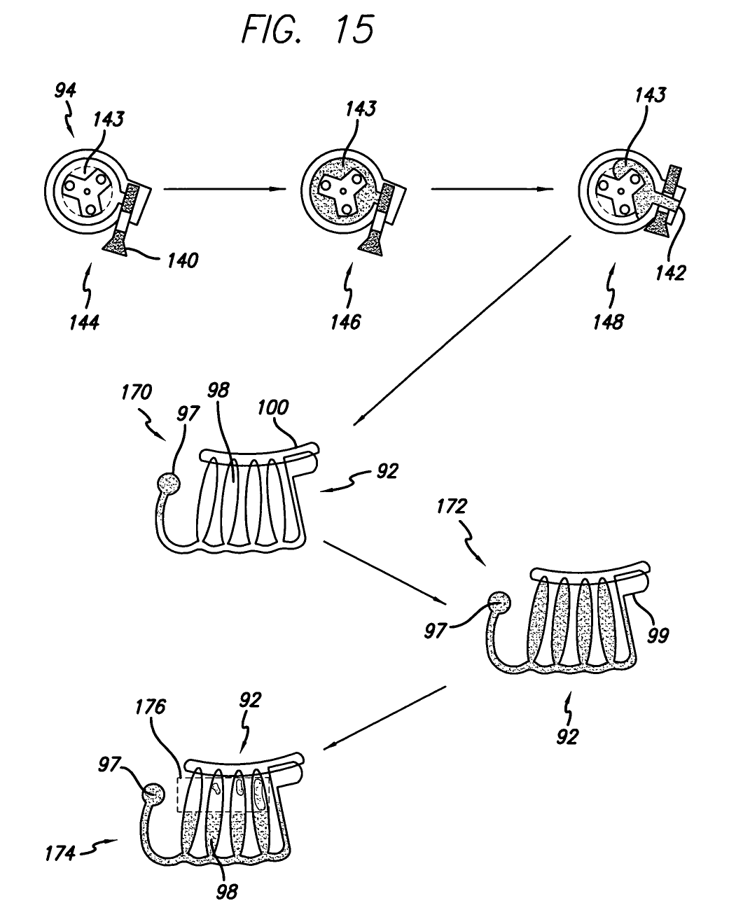

Each turbulent shear device 94 is positioned over a respective test unit 92 (FIG. 10D) such that the opening 142 in the shear device is directly above the application site 96 in the top layer 106 of the test unit (FIG. 11). The outflow of fluid is controlled by the valve mechanism 140 integrated with the shear device. The valve has at least two positions. When the valve is in a normally closed position 144, 146 (FIG. 15), the fluid is confined in the shear device. When the valve is in the open position 148 (FIG. 15), fluid is allowed to drain away from the shear device into the test unit through the sample application site 96. Filling 170, 172, 174 (FIG. 15) of the test unit reaction channels 98 can be accomplished with the aid of either centrifugal force or capillary action, or both. The opening and closing of the valve can be accomplished by linear (FIG. 15) or rotational actuation (not shown) methods. The actuation mechanism can be controlled by mechanical, opto-electrical, magnetic or electromagnetic means.

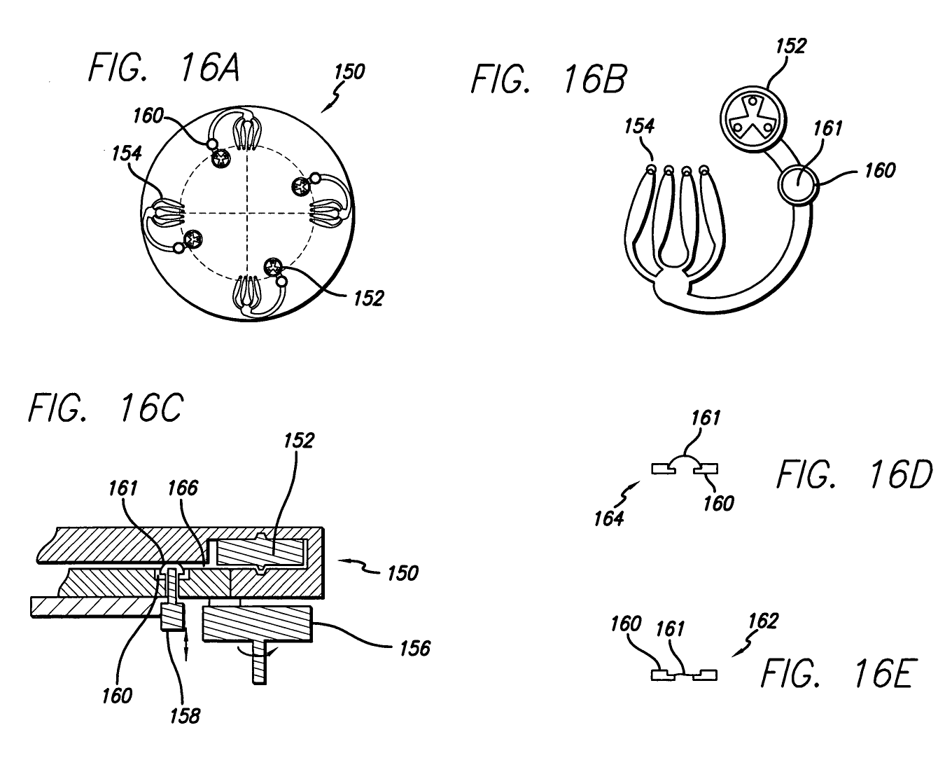

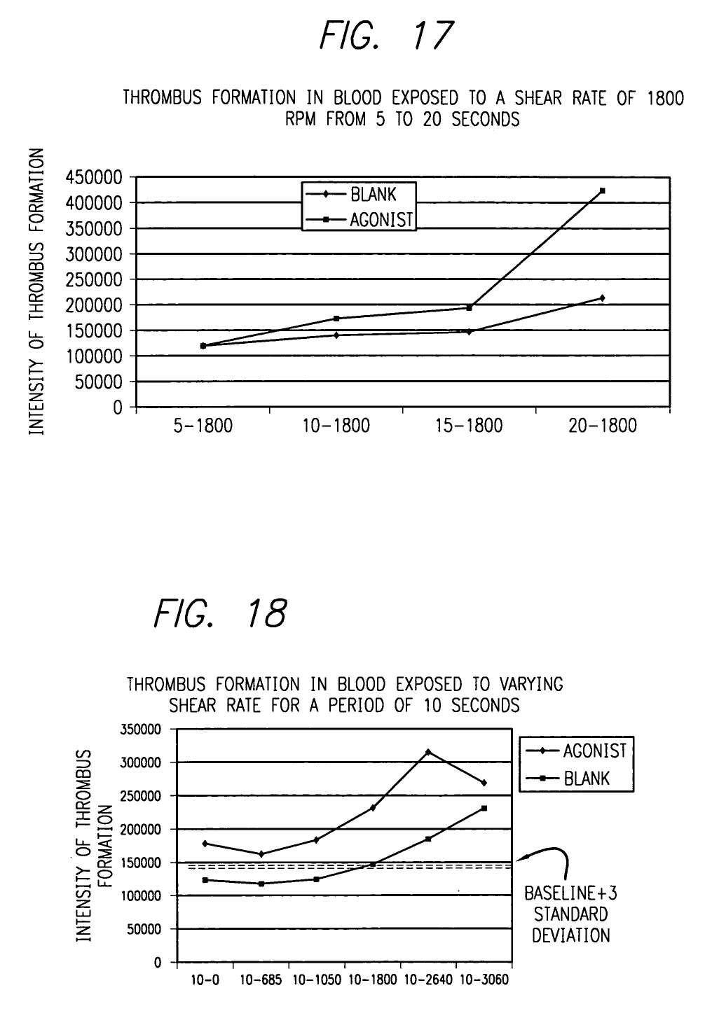

FIGS. 16A-16D illustrate an alternate design of a flow control mechanism in a test cartridge 150. In this embodiment, the valve 160 is associated with the test unit 154 instead of the shear device 152 (FIG. 16B). The valve mechanism 160 is located in the flow path downstream from the sample application site (not shown). A deformable membrane 161 controls the opening and closing of the flow path, and is configured from elastic materials or tape made out of nylon, polyester or other polymers. Displacement of an actuator 158 placed perpendicular to the membrane 161 (FIG. 16C) causes the membrane to deform 164 thereby narrowing the flow path (FIG. 16D). The flow path is restored when the actuator and the membrane are allowed to return to the resting state 162 (FIG. 16E). Actuation mechanism can be controlled by mechanical, opto-electrical, magnetic or electromagnetic means.

FIG. 15 illustrates the preferred mode of operation to conduct a thrombogenic test. At the beginning of a test 144, fifty to one hundred micro-liters (μL) of anti-coagulated sample is introduced into the shear device 94 with the valve 140 set at the closed position 146. For assessment of primary hemostasis, the sample is exposed to low or moderate shear stress (500 to 1500 rpm). For assessment of thrombotic risks or drug monitoring, the sample is exposed to moderate or high shear stress (up to 3400 rpm). The valve is opened at the end of the shear cycle 148 to allow outflow of sample into the application site 97 of the test unit 92. Blood is directed sequentially into the four reaction channels 98 by centrifugal force, capillary action or both 170, 172, 174. When the sample reaches the common vent area 100, sample flow stops. Preferably, the application site 97 is positioned at the level of the vent 100. The principle of operation of stop junctions is described in U.S. Pat. No. 5,230,866, incorporated herein by reference. Excess sample, if present, is drained into the by-pass channel 99. The sample rehydrates and mixes with the reagents pre-dried in the reaction channels 98 under the influence of a low centrifugal force 172. At the end of the mixing period, a sufficiently high centrifugal force is applied to separate plasma from the cellular particulates 174. This scheme allows separation of non-adherent cells from adherent thrombi and cell aggregates. Adherent thrombi and cell aggregates, if present, are readily observed in the plasma phase where chemical reaction occurs. The removal of non-adherent cellular content facilitates direct observation of adherent thrombi in the plasma phase in the view area 176.

In the ninth embodiment, pre-dried reagents may be used to provide a second triggering signal for platelets and/or leukocytes under low shear condition. For primary hemostasis studies, dried reagents may include various subtypes of collagen, adenosine diphosphate (ADP), ristocetin, and thrombin receptor activator, and the like. These substrates can be used individually or in combinations to activate cells. Samples that show low levels of thrombus formation as compared to normal levels of thrombus formation may indicate platelet dysfunction or other disorders in primary hemostasis.

In the tenth embodiment, a method is provided to assess thrombotic risks of a biologic sample. In the current invention, thrombogenic substrates are used to provide a second triggering signal for platelets and/or leukocytes in conjunction with moderate and high shear condition described in this invention. These substrates include substances that are commonly found in atherosclerotic plaques. Thrombogenic substrates may include tissue factor, collagen, lipo-proteins, chemotaxins, adhesion molecules, platelet and leukocyte membrane fragments that contain tissue factor or coagulation proteins, and the like. These substrates are presented as pre-dried reagents in reaction channels of a test cartridge. These reagents are provided in concentrations that induce only local formation of adherent aggregates and thrombi but do not trigger an overt clot formation that interferes with the separation of cells from plasma.

In the eleventh embodiment, inhibitory actions of pharmacological agents can be investigated during various stages of the thrombogenic test. Agents can be introduced into the biological fluid to study loading of drugs into cells prior to shear activation. Agents can also be added at the end of the shear activation to study inhibitory effects of agents on pre-activated cells. An agent or combinations of agents can be introduced in solution or in pre-dried format on the test cartridge. The test can be used to establish the loading does of a given agent that suppresses a desired level of adherent thrombi. The Thrombogenic Device is particularly well adapted for monitoring therapeutic effects of agents that may exhibit inhibitory or stimulatory effects on the formation of adherent thrombi.

In the twelfth embodiment, biological fluid may include either native or anti-coagulated whole blood. Citrate at 0.1 and 0.129 M at a ratio of nine parts sample to one part anticoagulant can be used with the current invention. For specimen that is older than six hours, 0.1 M buffered citrate is preferred for maintaining the pH level. When anti-coagulated whole blood is used, sufficient calcium ion is added to the sample to neutralize the effects of the anticoagulant and restore extracellular calcium ion to the physiological range. Calcium ion can be presented in either wet or dry format in the shear device or in the thrombogenic test unit.

### EXAMPLE 1

### Cell Activation by Turbulent Shear Device

Venous blood was collected from normal volunteers into one-tenth volume of 3.8% trisodium citrate. Six μL of one hundred twenty-five mM CaCl₂ was mixed with twelve μL of phosphate buffered saline and one hundred thirty-two μL of citrated blood in a five hundred μL polycarbinate reaction vessel. The reaction mixture was immediately transferred to shear devices and exposed to varying shear stress for a pre-determined amount of time. Shear rate ranging from five hundred rpm to three thousand rpm. Twenty μL of processed sample was immediately transferred onto the application site of a blank thrombogenic test cartridge. After the filling is complete, the test cartridge was allowed to incubate for ninety seconds. The cartridge was then centrifuged initially at 600 rpm for 80 seconds and immediately followed by a more rapid spin at eighteen hundred rpm for an additional one hundred sixty seconds. Non-sheared but re-calcified samples were used as controls. At the end of the spin cycle, test cartridges were observed using a light microscope at 40× magnification. The presence of thrombi in the reaction area was documented using a SPOT CCD camera.

FIG. 17 illustrates intensity of thrombus formation in whole blood exposed to a constant shear rate of eighteen hundred rpm over duration between five and twenty seconds. With a twenty second exposure at a rate of eighteen hundred rpm, significant amount of adherent thrombi were observed in all reaction areas. Higher reactivity was observed in areas where the shear-activated samples were further exposed to agonists. In this experiment, 0.5 μL of a 1:500 or 1:1000 dilution of recombinant tissue factor (Innovin, Dade) in phosphate buffered saline was pre-dried in the reaction areas on the test cartridge. These reagents are provided in concentrations that induce only local formation of adherent aggregates and thrombi, but do not trigger an overt clot formation that interferes with the separation of cells from plasma as described herein regarding the present invention.

In a separate experiment, re-calcified blood was exposed to shear rate ranging between five hundred to three thousand sixty rpm over a fixed exposure time of ten seconds. FIG. 18 illustrates the intensity of thrombus formation as a function of shear rate at a fixed ten second exposure time. Significant amounts of thrombi were generated at a shear rate of eighteen hundred rpm or higher. Higher reactivity was also observed across all categories where the shear-activated samples were further exposed to agonists.

### EXAMPLE 2

A first biologic sample from a patient with Glanzmann Thrombasthenia (GT) and a second biologic sample from a patient with von-Willebrand Disease were tested in the thrombogenic test system of the present invention using ADP or Ristocetin as agonists (Sigma). A third biologic sample from normal individual was used as control. None of these samples were exposed to shear stress. As illustrated in FIG. 19, thrombus formation was significantly lower with the two diseased patients (first and second samples) as compared to healthy normal control (third sample), suggesting platelet dysfunction or disorders in primary hemostasis.

### EXAMPLE 3

Thrombus formation in response to the challenge of shear stress and platelet agonists can be used as a test system to study agents that have stimulatory or inhibitory activity. To show a positive modulation, molecules such as fibrinogen, von-Willebrand factor, selecting, adhesion proteins and the like can be added to the blood sample prior to shear activation. To show negative modulation, antibodies that recognize coagulation proteins such as fibrinogen, von-Willebrand factor, adhesion molecules, tissue factor, and various factors in the intrinsic or extrinsic coagulation pathways can be added to the blood sample prior to shear activation. Alternatively, pharmaceutical agents with inhibitory effects on platelet or coagulation can be used in place of the abovementioned antibodies.

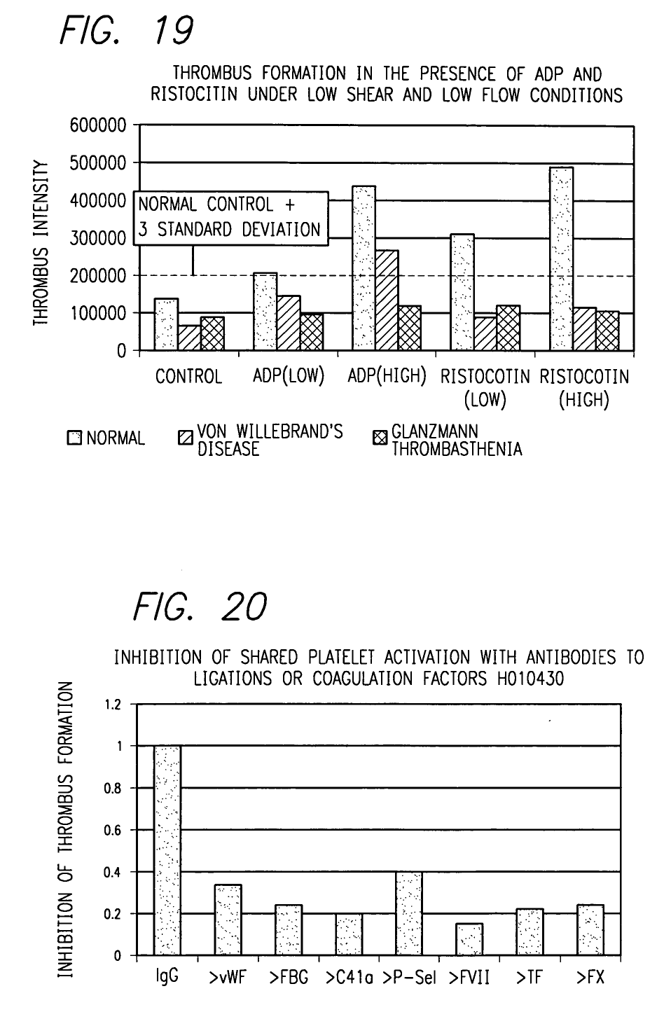

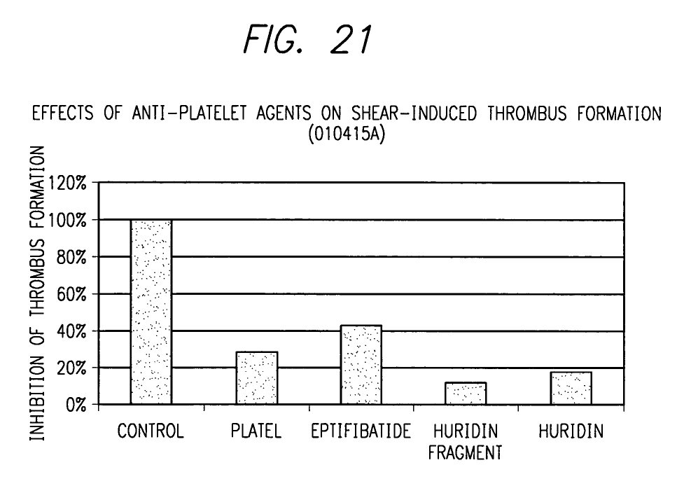

The result from tests using negative modulation on thrombus formation incorporating various antibodies to the coagulation system is shown in FIG. 20. Six μL of antibodies was added to six μL of one hundred twenty-five mM CaCl₂, six μL of phosphate buffered saline and one hundred thirty-two μL blood samples in a polystyrene container at room temperature. The mixture was immediately transferred to the shear unit and processed using the protocol stated in Example 1. All selected antibodies showed a greater than fifty percent inhibition of thrombus formation on the thrombogenic test device of the present invention.

FIG. 21 illustrates the inhibitory effects of various drugs on the formation of thrombus in sheared-activated whole blood. In general, six μL of diluted drug solution was mixed with six mL of phosphate buffered saline and one hundred thirty-two μL of citrated blood at room temperature. Total incubation time for Eptifibatide was sixty minutes and one hundred twenty minutes for Platel. Six mL of one hundred twenty-five mM CaCl₂ was added to the blood-drug mixtures immediately prior to shear activation. Final concentrations of the drug in the blood-drug mixtures were as follows: 0.004 μg/μl for Eptifibatide (COR Therapeutics, Inc.), 0.02 μg/μl for Platel (Otsuka Pharmaceuticals), 0.05 μg/μl for Hirudin (Sigma), and 0.05 μg/μl for Hirudin fragment 54-65 (Sigma).

While a particular form of the invention has been illustrated and described, it will also be apparent to those skilled in the art that various modifications can be made without departing from the scope of the invention. More specifically, it should be clear that the present invention is not limited to the disclosed methods and devices. Additionally, any of a variety of designs and applications of test devices and methods can benefit from the present invention. Further, the dimensions and materials referenced herein are by way of example only and not intended to be limiting. Accordingly, it is not intended that the invention be limited, except as by the appended claims.

What is claimed is:

1. A device for applying a shear stress to a biological fluid, comprising:
a housing; and
a cylinder rotatably disposed within the housing, the cylinder having sides with a non-uniform curvature.

2. The shear device of claim 1, wherein the cylinder has at least one arm containing a magnetic material.

3. The shear device of claim 2, wherein the cylinder has three arms and only two arms contain the magnetic material.

4. The shear device of claim 1, wherein the cylinder is disposed offset from a center axis of the housing.

5. The shear device of claim 1, wherein an outside wall of the cylinder or an inside wall of the housing contains obstructions.

6. The shear device of claim 1, wherein one of the surfaces of the housing is configured with a fitting for receiving a first fluid transfer device, and at least one other surface of the housing is configured with a fitting for receiving a second fluid transfer device.

7. A test cartridge for receiving a biological fluid, comprising:
a test unit including,
an opening for receiving fluid,
a transfer channel in fluid communication with the reservoir,
at least one reaction channel in fluid communication with the transfer channel, and
a reservoir interposed between the transfer channel and each reaction channel.

8. The test cartridge of claim 7, further comprising a second reservoir in fluid communication with the opening.

9. The test cartridge of claim 7, further comprising at least one vent associated with each reaction channel.

10. The test cartridge of claim 9, further comprising means for viewing a portion of each reaction channel.

11. The test cartridge of claim 7, further comprising at least one shear device of claim 1 in fluid communication with at least one reaction channel.

12. The test cartridge of claim 10, further comprising a plurality of reaction channels and a plurality of shear devices configured according to claim 3, wherein each shear device is in fluid communication with one of the reaction channels.

13. The test cartridge of claim 12, further comprising a plurality of valves, each valve interposed between an outlet of each shear device and the opening of each test unit.

14. The test cartridge of claim 13, wherein each valve is configured with a deformable membrane.

15. The test cartridge of claim 14, further comprising a bottom layer, a middle layer containing at least one reservoir, each reaction channel and a top layer containing each vent and configured to retain each shear device.

16. A method for analyzing a biological fluid sample, comprising:
providing a test cartridge according to claim 7;
providing a composition of matter in at least one reaction channel of the test cartridge;
providing a shear device according to claim 1;
applying a biological fluid sample to the shear device;
causing the cylinder of the shear device to rotate so as to apply a shear stress to the fluid sample;
transferring at least a portion of the fluid sample to the test cartridge;
applying a rotational force to the test cartridge so as to move a portion of the fluid sample into at least one reaction channel; and
analyzing the portion of the fluid sample within at least one reaction channel.

17. The method of claim 16, wherein providing a composition of matter includes applying a reagent selected from the group consisting of collagens, adenosine diphosphate, ristocetin, and thrombin receptor activators.

18. The method of claim 16, wherein providing a composition of matter includes applying a thrombogenic substrate selected from the group consisting of tissue factor, collagen, lipo-proteins, chemotaxins, adhesion molecules, and platelet or leukocyte membrane fragments containing tissue factor or coagulation proteins.

19. The method of claim 18, wherein applying a thrombogenic substrate includes providing a reagent in a concentration that induces formation of adherent aggregates and thrombi without triggering an overt clot formation, so as to allow separation of cells in the biological sample from plasma.

20. A method for analyzing a blood sample, comprising:
providing a test cartridge having a plurality of shear devices each including an inlet and an outlet, a plurality of test units each having an inlet, and a plurality of valves each in fluid communication with the outlet of one shear device and the inlet of one test unit,
wherein the shear device includes a sample chamber, a housing disposed over the sample chamber, a rotor disposed within the sample chamber and having sides with a non-uniform curvature, the rotor further having at least one arm containing a magnetic material,
wherein each test unit includes a first reservoir in fluid communication with the inlet of the test unit, a transfer channel in fluid communication with the reservoir, a plurality of reaction channels in fluid communication with the transfer channel, a dried reagent in at least one reaction channel, and a viewing area associated with at least one reaction channel;
applying a blood sample to the shear device;
applying a magnetic force to the shear device to cause the rotor to move so as to apply a shear stress to the blood sample;
applying a rotational force to the test cartridge so as to move a portion of the blood sample from the shear device into at least one reaction channel; and
optically analyzing the portion of the blood sample within at least one reaction channel.
